(19) United States
(12) Patent Application Publication
(10) Pub. No.: US 2017/0273893 A1
(43) Pub. Date: Sep. 28, 2017

(54) AGENT FOR TEMPORARY SHAPING OF KERATIN-CONTAINING FIBERS HAVING A MIXTURE OF CONDITIONING POLYMERS

(71) Applicant: Henkel AG & Co. KGaA, Duesseldorf (DE)

(72) Inventors: Jisook Baek, Hamburg (DE); Ana Puls, Winsen (Luhe) (DE)

(73) Assignee: Henkel AG & Co. KGaA, Duesseldorf (DE)

(21) Appl. No.: 15/622,413

(22) Filed: Jun. 14, 2017

Related U.S. Application Data
(63) Continuation of application No. PCT/EP2015/079483, filed on Dec. 11, 2015.

(30) Foreign Application Priority Data
Dec. 18, 2014 (DE) 102014226410.3

(51) Int. Cl. A61K 8/894; A61K 8/81; A61K 8/67; A61K 8/41; A61Q 5/06; A61K 8/73; A61Q 5/12; A61K 8/34

(52) U.S. Cl. CPC A61K 8/894; A61Q 5/12; A61K 8/8182; A61K 8/345; A61K 8/416; A61Q 5/06; A61K 8/737; A61K 8/731; A61K 8/8176; A61K 8/673; A61K 2800/262; A61K 2800/594; A61K 2800/5426; A61K 2800/5422

(57) ABSTRACT

A transparent cosmetic composition and related methods are provided. The composition includes, in relation to the total weight thereof, 0.1 to 30 wt. % of a mixture of at least two conditioning polymers, 0.01 to 25 wt. % of a polyether-modified polysiloxane, 0.001 to 15 wt. % of a film-forming polymer, and 1 to 90 wt. % water.

### FIELD OF THE INVENTION

The present invention generally relates to the technical field of temporary shaping of keratin-containing fibers, in particular human hair.

### BACKGROUND OF THE INVENTION

Styling agents for shaping keratin-containing have been known for a long time and are used in various forms for styling, refreshing and fixing hairstyles which in many hair types can be obtained only by using stabilizing active ingredients. Both hair-treatment agents which serve to provide a permanent and also those which provide a temporary shape to the hair play an important role.

Sprayable products as well as products which can be worked into the hairstyle with the help of a comb or the fingers are suitable for temporary shaping of keratin-containing fibers. The latter-named product group comprises oils as well as gels, creams and powders.

Frizzy hair is one of the biggest problems which occurs if hair is subjected to high air humidity. This problem occurs most markedly in people with curly hair and leads to what is known colloquially as a “bad hair day”. In such a case, the hair loses its natural shape and/or its curl retention.

In people with naturally curly hair or with perms, often the problem occurs that the curls fall out and lose their bounce over time. This happens if, for example, the hair-treatment agents used, such as shampoos, rinses or styling agents, weigh down the hair.

Unlike straight hair, curly hair does not reflect as much light, whereby it shines a little less than normal.

A composition for improving the shine, styleability and curl retention and curl separation of curly hair is described in EP1741470 B1. This composition includes a film-forming polymer, a polyol in a quantity of more than 5 wt. % and polyacryloyldimethyl taurate and/or the salts thereof.

A further problem in the formulation of styling agents consists in the production of clear transparent compositions, as the polymers normally used in hair-styling agents often result in milky-white compositions. A milky appearance can, however, give the user the impression that the formulation can be clearly visible in the hair, thus reducing user acceptance of the styling agent.

The object of the present invention was therefore to make available transparent hair-treatment agents for temporary shaping of curly hair which give the hair a long-lasting, curly appearance, high volume, high shine and easy styleability.

### BRIEF SUMMARY OF THE INVENTION

A first subject matter of the present invention is transparent cosmetic compositions, including, in relation to the total weight thereof
a) 0.1 to 30 wt. % of a mixture of at least two conditioning polymers, wherein a conditioning polymer is selected from the group consisting of cationic guar compounds and poly(methacryloyloxyethyl trimethylammonium chloride) and mixtures thereof,
b) 0.01 to 25 wt. % of a polyether-modified polysiloxane,
c) 0.001 to 15 wt. % of a film-forming polymer and
d) 1 to 90 wt. % water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A long-lasting, curly appearance is characterized by a high curl retention and/or high bounce of the curls and/or high curl separation. The latter means that the curls can be clearly identified in a strand of hair.

It has been established that these objects can be achieved with the help of a combination of at least two conditioning polymers, an ethoxylated polyorganosiloxane and a film-forming polymer.

The composition is present in the form of a gel which is preferably applied directly with the hand or a comb, in the form of a viscous lotion, cream or paste, in the form of a wax, in the form of a sprayable liquid which is sprayed using a mechanical device, or in the form of a foamable liquid, which is expanded before application. Preferably the compositions are present in the form of a gel.

Corresponding hair-treatment agents are called hairsprays, hair gels, hair wax, hair mousse, hair-setting lotions or hair lotions. Preferred cosmetic compositions are hair gels.

If the compositions are present in the form of gels, creams, pastes or waxes, the viscosity of the compositions is preferably between 70,000 and 600,000 mPas, preferably between 100,000 and 400,000 mPas (20° C., rotation viscometer DV 2T “Brookfield”, TE spindle, 5 rotations per minute and Helipath). By clear transparent compositions are understood in the scope of the present invention, compositions which appear clear to the naked eye when on the surface of the hand. At a transmission measurement of 600 nm and at room temperature, these compositions have a transmission value of >93%.

A first essential constituent of the cosmetic composition is a mixture of least two conditioning polymers. A conditioning polymer is inevitably selected from the group consisting of cationic guar compounds, poly(methacryloyloxyethyl trimethylammonium chloride) and mixtures thereof.

Conditioning polymers are polymers which are applied to the hair and exert a conditioning effect there. In order to improve absorption behavior, conditioning polymers generally have cationic charges and are accordingly cationic or amphoteric polymers.

Guar is a polysaccharide which is obtained from the seeds of guar beans. Cationic guar compounds are mainly quaternized guar compounds which are obtained by reacting polysaccharides with trimethylammonium-substituted epoxides.

Suitable cationic guar compounds are marketed under the trade name Jaguar and have the INCI name Guar Hydroxypropyltrimonium Chloride. Furthermore, particularly suitable cationic guar compounds are also commercially available from the company Hercules under the name N-Hance®. Further cationic guar compounds are marketed by BASF SE under the name Cosmedia®. A preferred cationic guar compound is the commercial product AquaCat® from Hercules. This raw material is an already pre-dissolved cationic guar compound.

Poly(methacryloyloxyethyl trimethylammonium chloride), which can also be cross-linked, is a homopolymer which is also known under INCI name Polyquaternium-37. Such products are for example commercially available under the names Cosmedia® CTH or Cosmedia® Ultragel 300 (BASF SE) or Synthalen® CR (3V Group).

The homopolymer is preferably used in the form of a non-aqueous polymer dispersion. Such polymer dispersions are commercially available under the names Salcare° SC 95 and Salcare® SC 96. A polymer dispersion which is marketed under the name Cosmedia® Triple C (ex BASF SE) is also suitable.

The further conditioning polymers can be homo- or copolymers or polymers based on natural or synthetic polymers. The conditioning polymers can be cationic or amphoteric. The conditioning polymers include quaternary nitrogen groups which are included either in the polymer chain or preferably as a substituent at one or more of the monomers. Suitable cationic monomers are unsaturated, radically polymerizable compounds which have at least one cationic group, in particular ammonium-substituted vinyl monomers such as for example trialkyl methacryloxy alkylammonium, trialkyl acryloxy alkylammonium, dialkyl diallyl ammonium and quaternary vinylammonium monomers with cyclic, cationic nitrogen-containing groups such as pyridinium, imidazolium or quaternary pyrrolidones, for example alkylvinyl imidazolium, alkylvinyl pyridinium, or alkylvinyl pyrrolidone salts. The alkyl groups of these monomers are preferably lower alkyl groups such as for example C1 to C7 alkyl groups, particularly preferably C1 to C3 alkyl groups.

The monomers having ammonium groups can be copolymerized with non-cationic monomers. Suitable comonomers are for example acrylamide, methacrylamide; alkyl- and dialkyl acrylamide, alkyl- and dialkylmethacrylamide, alkylacrylate, alkylmethacrylate, vinylcaprolactone, vinylcaprolactam, vinylpyrrolidone, vinylester, for example vinylacetate, vinylalcohol, propylene glycol or ethylene glycol, wherein the alkyl groups of these monomers are preferably C1 to C7 alkyl groups, particularly preferably C1 to C3 alkyl groups.

There have proved particularly advantageous, from the large number of these polymers:

homopolymers of general formula —{CH₂—[CR¹COO—(CH₂)ₘN⁺R²R³R⁴]}ₙ X⁻,

in which R¹=—H or —CH₃, R², R³ and R⁴ are selected, independently of one another, from C1-4 alkyl, alkenyl or hydroxyalkyl groups, m=1, 2, 3 or 4, n is a natural number and X⁻ is a physiologically compatible organic or inorganic anion. In the scope of these polymers, those ones are preferred for which at least one of the following conditions applies: R¹ stands for a methyl group, R², R³ and R⁴ stand for methyl groups, m has the value 2.

For example halogenide ions, sulfate ions, phosphate ions, methosulfate ions and organic ions such as lactate, citrate, tartrate and acetate ions come into consideration as physiologically compatible counterions X⁻. Methosulfates and halogenide ions, in particular chloride, are preferred.

Suitable cationic, conditioning polymers are for example copolymers according to the formula (Copo).

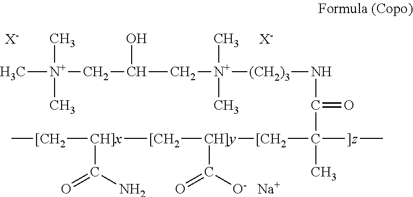

in which:

x+y+z=Q

Q stands for values of from 3 to 55,000, preferably of from 10 to 25,000, particularly preferably of from 50 to 15,000, further preferably of from 100 to 10,000, even more preferably of from 500 to 8000 and in particular of from 1000 to 5000,
x stands for (0 to 0.5) Q, preferably for (0 to 0.3) Q and in particular for the values 0, 1, 2, 3, 4, 5, wherein the value is preferably 0,
y stands for (0.1 to 0.95) Q, preferably for (0.5 to 0.7) Q and in particular for values of from 1 to 24,000, preferably of from 5 to 15,000, particularly preferably of from 10 to 10,000 and in particular of from 100 to 4800,
z stands for (0.001 to 0.5) Q, preferably for (0.1 to 0.5) Q and in particular for values of from 1 to 12,500, preferably of from 2 to 8000, particularly preferably of from 3 to 4000 and in particular of from 5 to 2000.

Regardless of which of the preferred copolymers of formula (Copo) is/are used, cosmetic compositions which are characterized in that the ratio of (y:z) is 4:1 to 1:2, preferably 4:1 to 1:1, are preferred.

Regardless of which copolymers are used in the compositions, cosmetic compositions in which the copolymer has a molar mass of from 10,000 to 20 million gmol⁻¹, preferably of from 100,000 to 10 million gmol⁻¹, more preferably of from 500,000 to 5 million gmol⁻¹ and in particular of from 1.1 million to 2.2 million gmol⁻¹, are preferred.

A highly preferred copolymer which is structured as shown previously is commercially available under the name Polyquaternium-74.

Suitable cationic, conditioning polymers which are derived from natural polymers are cationic derivatives of polysaccharides, for example cationic derivatives of cellulose or starch. Furthermore, chitosan and chitosan derivatives are suitable.

Cationic polysaccharides have the general formula G-O-B-N+RₐR_{b}R_{c} A⁻
G is an anhydroglucose residue, for example starch or cellulose anhydroglucose;
B is a divalent compound group, for example alkylene, oxyalkylene, polyoxyalkylene or hydroxyalkylene;
Rₐ, R_{b} and R_{c} are, independently of one another, alkyl, aryl, alkylaryl, arylalkyl, alkoxyalkyl or alkoxyaryl each with up to 18 C atoms, wherein the total number of C atoms in Rₐ, R_{b} and R_{c} is preferably at most 20;
A⁻ is a customary counterion and is preferably chloride.

Cationic, i.e. quaternized, celluloses are commercially available with different degrees of substitution, cationic charge density, nitrogen content and molecular weights. For example, Polyquaternium-67 is commercially available under the names SoftCat® polymer SL or SoftCat® polymer SK (Dow).

Further cationic celluloses are Ucare® polymer JR 400 (Dow, INCI name Polyquaternium-10) and polymer Quatrisoft® LM-200 (Dow, INCI name Polyquaternium-24). Further commercial products are the compounds Celquat® H 100 and Celquat® L 200.

A suitable chitosan is marketed for example from Kyowa Oil&Fat, Japan, under the trade name Flonac®. A preferred chitosan salt is chitosonium pyrrolidone carboxylate which is marketed for example under the name Kytamer® PC from Amerchol, USA. Further chitosan derivatives are freely commercially available under the trade names Hydagen® CMF, Hydagen® HCMF and Chitolam® NB/101.

A further group of outstandingly usable cationic, conditioning polymers are polymers based on glucose. The following figure shows such a cationic alkyl oligoglucoside.

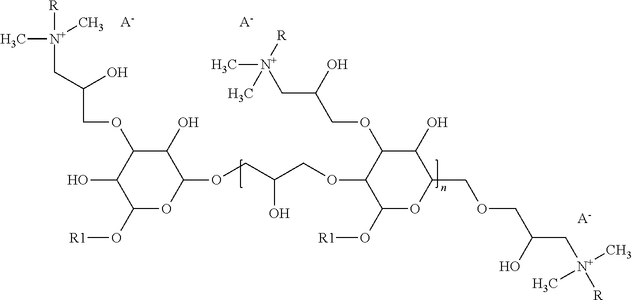

In the above-shown formula, the residues R stand, independently of one another, for a linear or branched C6 to C30 alkyl residue, a linear or branched C6-C30 alkenyl residue, preferably the residue R stands for a residue R selected from: lauryl, myristyl, cetyl, stearyl, oleyl, behenyl or arachidyl.

The residues R1 stand, independently of one another, for a linear or branched C6 to C30 alkyl residue, a linear or branched C6 to C30 alkenyl residue, preferably the residue R stands for a residue R selected from: butyl, capryl, caprylyl, octyl, nonyl, decanyl, lauryl, myristyl, cetyl, stearyl, oleyl, behenyl or arachidyl. Particularly preferably, the residues R1 are identical. The residues R1 are even more preferably selected from technical mixtures of fatty alcohol cuts of C6/C8 fatty alcohols, C8/C10 fatty alcohols, C10/C12 fatty alcohols, C12/C14 fatty alcohols, C12/C18 fatty alcohols, and highly preferably those technical fatty alcohol cuts which are of plant origin. The counterion for cationic charge is a physiologically compatible anion, for example halogenide, methosulfate, phosphate, citrate, tartrate, etc. The counterion is preferably a halogenide, such as fluoride, chloride, bromide or methosulfate. The anion chloride is highly preferred.

Particularly preferred examples of the cationic alkyl oligoglucosides are the compounds with INCI names Polyquaternium-77, Polyquaternium-78, Polyquaternium-79, Polyquaternium-80, Polyquaternium-81 and Polyquaternium-82. The cationic alkyl oligoglucosides with the names Polyquaternium-77, Polyquaternium-81 and Polyquaternium-82 are highly preferred.

Such compounds, for example under the name Poly Suga® Quat from Colonial Chemical Inc., may be available.

Self-evidently it is also included that more mixtures of cationic alkyl oligoglucosides can be used. In this case it is preferred if respectively a long-chain and a short-chain cationic alkyl oligoglucoside can preferably be used simultaneously.

A further preferred cationic, conditioning polymer can be obtained on the basis of ethanol amine. The polymer is commercially available under the name Polyquaternium-71.

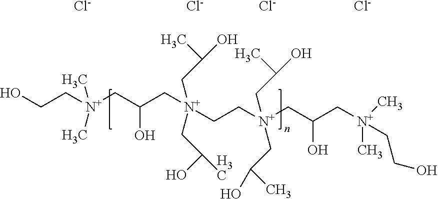

For example, this polymer can be used under the name Cola® Moist 300 P from Colonial Chemical Inc.

Furthermore, particularly preferably, a cationic alkyl oligoglucoside, as shown in the following figure, can be used.

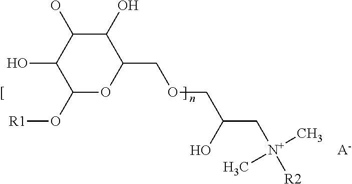

In the above-shown formula, the residue R2 stands, independently of one another, for a linear or branched C6 to C30 alkyl residue, a linear or branched C6-C30 alkenyl residue, preferably the residue R stands for a residue R selected from: lauryl, myristyl, cetyl, stearyl, oleyl, behenyl or arachidyl.

The residue R1 stands for a linear or branched C6 to C30 alkyl residue, a linear or branched C6 to C30 alkenyl residue, preferably the residue R1 stands for a residue R selected from: butyl, capryl, caprylyl, octyl, nonyl, decanyl, lauryl, myristyl, cetyl, stearyl, oleyl, behenyl or arachidyl. The residue R1 is even more preferably selected from technical mixtures of fatty alcohol cuts of C6/C8 fatty alcohols, C8/C10 fatty alcohols, C10/C12 fatty alcohols, C12/C14 fatty alcohols, C12/C18 fatty alcohols, and highly preferably those technical fatty alcohol cuts which are of plant origin. The index n stands for a number between 1 and 20, preferably between 1 and 10, more preferably between 1 and 5 and highly preferably between 1 and 3. The counterion for cationic charge, A⁻, is a physiologically compatible anion, for example halogenide, methosulfate, phosphate, citrate, tartrate, etc. The counterion is preferably a halogenide, such as fluoride, chloride, bromide or methosulfate. The anion chloride is highly preferred.

Particularly preferred examples of the cationic alkyl oligoglucosides are the compounds with INCI names Laurdimoniumhydroxypropyl Decylglucosides Chloride, Laurdimoniumhydroxypropyl Laurylglucosides Chloride, Stearyldimoniumhydroxypropyl Decylglucosides Chloride, Stearyldimoniumhydroxypropyl Laurylglucosides Chloride, Stearyldimoniumhydroxypropyl Laurylglucosides Chloride or Cocoglucosides Hydroxypropyltrimonium Chloride.

Such compounds, for example under the name Suga® Quat from Colonial Chemical Inc., may be available.

Self-evidently it is also included that more mixtures of cationic alkyl oligoglucosides can be used. In this case it is preferred if respectively a long-chain and a short-chain cationic alkyl oligoglucoside can preferably be used simultaneously.

A further preferred cationic, conditioning polymer comprises at least one structural unit of formula (I), at least one structural unit of formula (II), at least one structural unit of formula (III) and at least one structural unit of formula (IV),

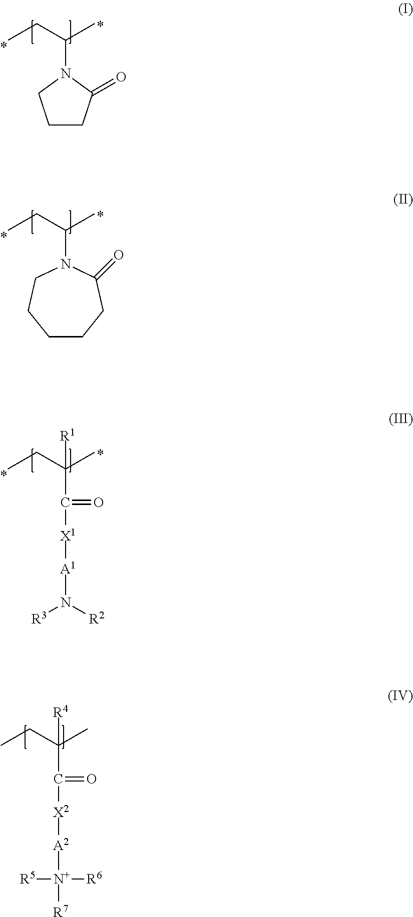

wherein
R¹ and R⁴ stand, independently of one another, for a hydrogen atom or a methyl group,
X¹ and X² stand, independently of one another, for an oxygen atom or a group NH,
A¹ and A² stand, independently of one another, for a group ethane-1,2-diyl, propane-1,3-diyl or butane-1,4-diyl, R², R³, R⁵ and R⁶ stand, independently of one another, for a (C₁ to C₄) alkyl group, R⁷ stands for a (C₈ to C₃₀) alkyl group.

According to the above formulae and all subsequent formulae, a chemical bond which is characterized with the symbol * stands for a free valence of the corresponding structural fragment.

All possible physiologically compatible anions, such as for example chloride, bromide, hydrogen sulfate, methyl sulfate, ethyl sulfate, tetrafluoroborate, phosphate, hydrogen phosphate, dihydrogen phosphate or p-toluene sulfonate, triflate, serve to compensate the positive polymer charge in the agent. Examples of (C₁ to C₄) alkyl groups according to the invention are methyl, ethyl, propyl, isopropyl, butyl, sec-butyl, isobutyl, tert-butyl.

Examples of (C₈ to C₃₀) alkyl groups according to the invention are octyl (capryl), decyl (caprinyl), dodecyl (lauryl), tetradecyl (myristyl), hexadecyl (cetyl), octadecyl (stearyl), eicosyl (arachyl), docosyl (behenyl).

Subsequent cationic, conditioning polymers can be used in the agents if, in respect of the aforementioned formulae (I) to (IV), the conditioning polymers have one or more of the following features:
R¹ and R⁴ each stand for a methyl group,
X¹ stands for a group NH,
X² stands for a group NH,
A¹ and A² stand, independently of one another, for ethane-1,2-diyl or propane-1,3-diyl,
R², R³, R⁵ and R⁶ stand, independently of one another, for methyl or ethyl, (particularly preferably for methyl),
R⁷ stands for a (C₁₀ to C₂₄) alkyl group, in particular for decyl (caprinyl), dodecyl (lauryl), tetradecyl (myristyl), hexadecyl (cetyl), octadecyl (stearyl), eicosyl (arachyl) or docosyl (behenyl).

It is preferred to select the structural unit of formula (III) from at least one structural unit of formula (III-1) to (III-8).

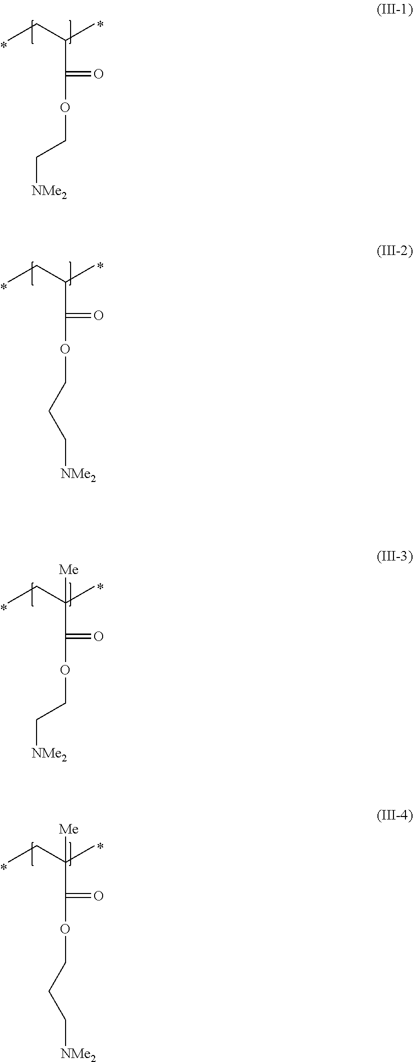

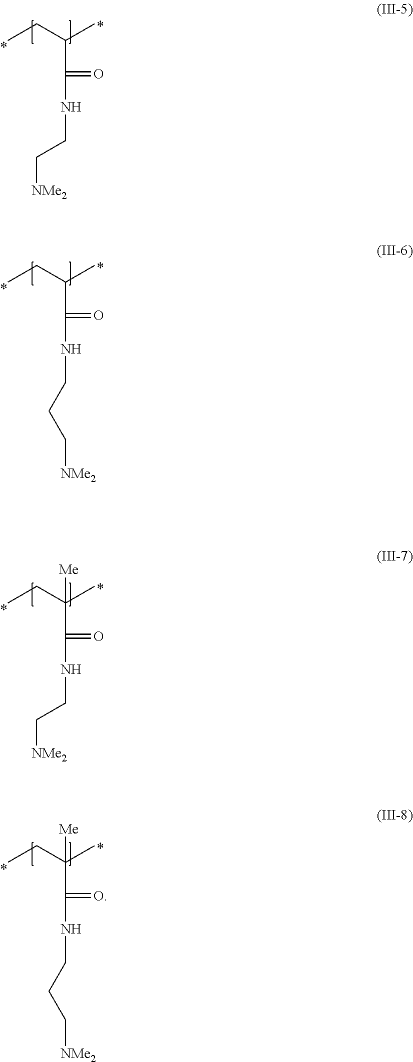

Moreover, it has proved particularly preferable to choose the structural unit according to formula (III-7) and/or formula (III-8) as the structural unit of formula (III). The structural unit of formula (III-8) is a quite particularly preferred structural unit.

Furthermore, with a view to achieving the object, it is preferable if the structural unit of formula (IV) is selected from at least one structural unit of formulae (IV-1) to (IV-8)

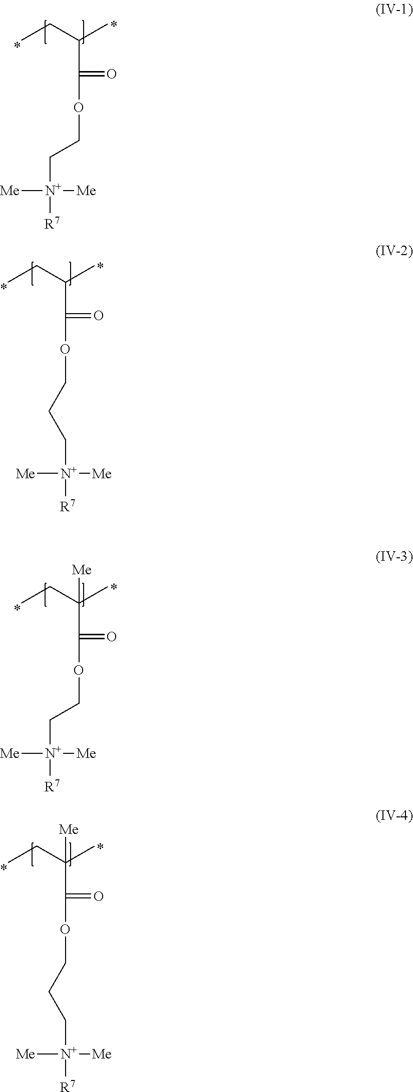

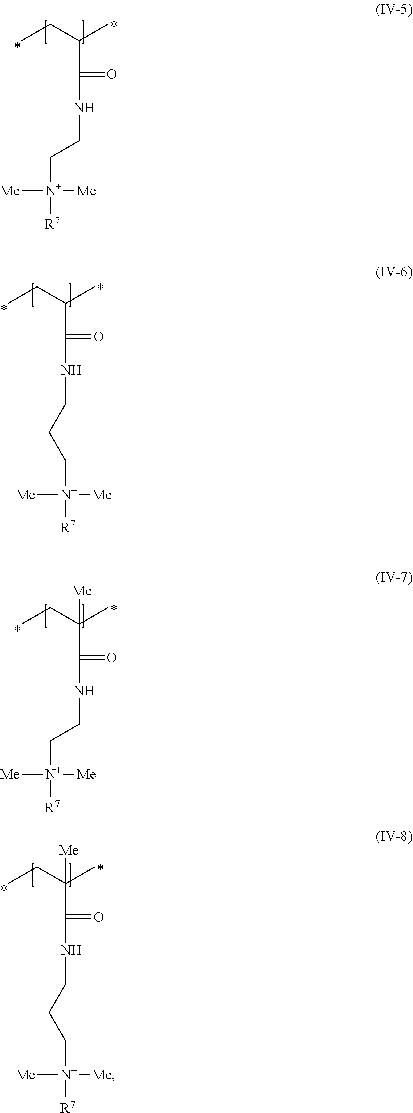

wherein in each case R⁷ stands for a (C₆ to C₃₀) alkyl group.

In turn, the structural units of formula (IV-7) and/or formula (IV-8), in which respectively R⁷ stands for octyl (capryl), decyl (caprinyl), dodecyl (lauryl), tetradecyl (myristyl), hexadecyl (cetyl), octadecyl (stearyl), eicosyl (arachyl) or docosyl (behenyl) are the particularly preferred structural unit of formula (IV). The structural unit of formula (IV-8) is a quite particularly preferred structural unit of formula (IV).

A cationic, conditioning polymer which is quite particularly preferably included in the agent comprises at least one structural unit of formula (I), at least one structural unit of formula (II), at least one structural unit of formula (III-8) and at least one structural unit of formula (IV-8),

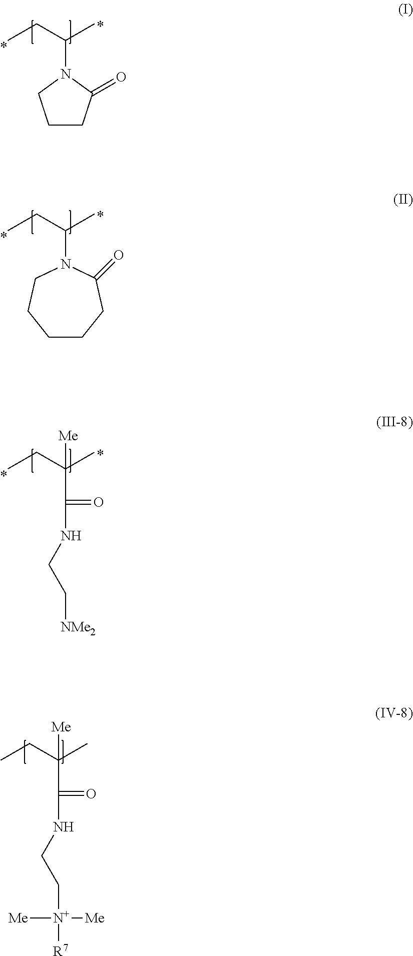

wherein R⁷ stands for octyl (capryl), decyl (caprinyl), dodecyl (lauryl), tetradecyl (myristyl), hexadecyl (cetyl), octadecyl (stearyl), eicosyl (arachyl) or docosyl (behenyl).

A quite particularly preferred cationic, conditioning polymer is the copolymer of N-vinylpyrrolidone, N-vinylcaprolactam, N-(3-dimethylaminopropyl)methacrylamide and 3-(methacryloylamino)propyl lauryl dimethyl ammonium chloride (INCI name: Polyquaternium-69) which for example is marketed under the trade name AquaStyle® 300 (28-32 wt. % active substance in ethanol-water mixture, molecular weight 350,000), from ISP.

Further preferred cationic, conditioning polymers are for example
cationized honey, for example the commercial product Honeyquat® 50,
polymeric dimethyldiallyl ammonium salts and the copolymers thereof with esters and amides of acrylic acid and methacrylic acid. The products commercially available under the names Merquat® 100 (poly(dimethyldiallyl ammonium chloride)) and Merquat® 550 (dimethyldiallyl ammonium chloride acrylamide copolymer) are examples of such cationic conditioning polymers with INCI name Polyquaternium-7,
vinylpyrrolidone vinylimidazolium methochloride copolymers are available under the names Luviquat® FC 370, FC 550 and INCI name Polyquaternium-16, as well as FC 905 and HM 552,
quaternized polyvinyl alcohol,
as well as polymers with quaternary nitrogen atoms in the polymer main chain under the names Polyquaternium-2, Polyquaternium-17, Polyquaternium-18 and Polyquaternium-27,
vinylpyrrolidone-vinylcaprolactam acrylate terpolymers, as are commercially available under the name Aquaflex® SF 40, as third monomeric component with acrylic acid esters and acrylic acid amides for example.

Suitable amphoteric, conditioning polymers are such polymerizates in which a cationic group is derived from at least one of the following monomers:
(i) monomers with quaternary ammonium groups of general formula (Mono1),

R¹-CH═CR²—CO-Z-(CₙH₂ₙ)—N⁽⁺⁾R²R³R⁴ A⁽⁻⁾ (Mono1)
in which R¹ and R², independently of one another, stand for hydrogen or a methyl group and R³, R⁴ and R⁵, independently of one another, stand for alkyl groups with 1 to 4 carbon atoms, Z is an NH group or an oxygen atom, n is an integer of from 2 to 5 and A⁽⁻⁾ is the anion of an organic or inorganic acid,
(ii) monomers with quaternary ammonium groups of general formula (Mono2),

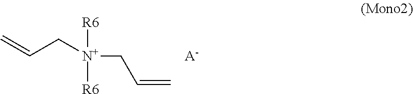

wherein R⁶ and R⁷, independently of one another, stand for a (C₁ to C₄) alkyl group, in particular for a methyl group and
A⁻ is the anion of an organic or inorganic acid,
(iii) monomeric carboxylic acids of general formula (Mono3),

R⁸-CH═CR⁹—COOH (Mono3)
in which R⁸ and R⁹, independently of one another, are hydrogen or methyl groups.

Such polymers in which monomers of type (i) are used, in which R³, R⁴ and R⁵ are methyl groups, Z is an NH group and A⁽⁻⁾ is a halogenide, methoxy sulfate or ethoxy sulfate ion; acrylamidopropyl trimethyl ammonium chloride is a particularly preferred monomer (i), are preferred. Preferably, acrylic acid is used as monomer (ii) for the named polymerizates.

Particularly preferred amphoteric, conditioning polymers are copolymers of at least one monomer (Mono1) or (Mono2) with monomer (Mono3), in particular copolymers from monomers (Mono2) and (Mono3). Quite particularly preferably used amphoteric polymers are copolymerizates of diallyl dimethyl ammonium chloride and acrylic acid. These copolymers are marketed under INCI name Polyquaternium-22, inter alia with the trade name Merquat® 280 (ex Lubrizol).

Additionally, the amphoteric, conditioning polymers in addition to a monomer (Mono1) or (Mono2) and a monomer (Mono3) can also include a monomer (Mono4) (I) monomeric carboxylic acid amides of general formula (Mono4),

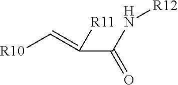

in which R¹⁰ and R¹¹, independently of one another, are hydrogen or methyl groups and R¹² stands for a hydrogen atom or a (C1 to C₈) alkyl group.

Quite particularly preferably usable amphoteric, conditioning polymers based on a comonomer (Mono4) are terpolymers of diallyl dimethyl ammonium chloride, acrylamide and acrylic acid. These copolymerizates are marketed under INCI name Polyquaternium-39, inter alia with the trade name Merquat® Plus 3330 (ex Lubrizol).

The amphoteric, conditioning polymers can generally be used both directly and also in the form of salt which is obtained by neutralizing the polymerizates, for example with an alkali hydroxide.

The polymers described thus far represent only a part of the usable conditioning polymers. In order not to have to describe all suitable cationic and/or amphoteric polymers together with their composition, in summary the INCI declarations of the preferred polymers are indicated. The preferred polymers have the INCI name: Polyquaternium-2, Polyquaternium-4, Polyquaternium-6, Polyquaternium-7, Polyquaternium-10, Polyquaternium-11, Polyquaternium-15, Polyquaternium-16, Polyquaternium-17, Polyquaternium-18, Polyquaternium-22, Polyquaternium-24, Polyquaternium-28, Polyquaternium-32, Polyquaternium-33, Polyquaternium-34, Polyquaternium-35, Polyquaternium-37, Polyquaternium-39, Polyquaternium-41, Polyquaternium-42, Polyquaternium-44, Polyquaternium-46, Polyquaternium-47, Polyquaternium-55, Polyquaternium-67, Polyquaternium-68, Polyquaternium-69, Polyquaternium-72, Polyquaternium-74, Polyquaternium-76, Polyquaternium-86, Polyquaternium-89, Polyquaternium-95 and Polyquaternium-101 and mixtures thereof.

Of the named polymers, particularly preferably Polyquaternium-11, Polyquaternium-46, Polyquaternium-72 or a mixture of two or three of the named polymers are used as conditioning polymer in combination with a cationic guar compound and/or poly(methacryloyloxyethyl trimethylammonium chloride) in the cosmetic compositions.

Polyquaternium-11 is the reaction product of diethyl sulfate with a copolymer of vinylpyrrolidone and dimethylaminoethyl methacrylate. Suitable commercial products can be obtained for example under the names Dehyquart® CC 11 and Luviquat® PQ 11 PN from BASF SE or Gafquat 440, Gafquat 734, Gafquat 755 or Gafquat 755N from Ashland Inc. Polyquaternium-11 is preferably used in a quantity of from 1 to 10 wt. % in relation to the total weight of the cosmetic composition. It is quite particularly preferred that Polyquaternium-11 is used in combination with a cationic guar compound.

Polyquaternium-46 is the reaction product of vinylcaprolactam and vinylpyrrolidone with methyl vinyl imidazolium methosulfate and can for example be obtained under the name Luviquat® Hold from BASF SE. Polyquaternium-46 is preferably used in a quantity of from 1 to 5 wt. % in relation to the total weight of the cosmetic composition. It is quite particularly preferred that Polyquaternium-46 is used in combination with a cationic guar compound. It is indeed highly preferred that Polyquaternium-46 is used in combination with a cationic guar compound and Polyquaternium-11.

A particularly preferably usable cationic, conditioning polymer is Polyquaternium-72. Polyquaternium-72 is a special cellulose. This cellulose is a hydroxyethyl cellulose which has been quaternized. Polyquaternium-72 is a trimonium and cocodimonium hydroxyethyl cellulose. Polyquaternium-72 can be used both in solid form and also pre-dissolved already in aqueous solution. The use for example of the commercial product Mirustyle® CP from Croda is preferred. The cosmetic compositions include Polyquaternium-72 preferably in a quantity of from at least 0.5 wt. % in relation to the total weight of the cosmetic compositions. Polyquaternium-72 is preferably used in a quantity of from 1 to 10 wt. % in relation to the total weight of the cosmetic composition. It is quite particularly preferred that Polyquaternium-72 is used in combination with poly(methacryloyloxyethyl trimethylammonium chloride).

The total quantity of conditioning polymers in relation to the total weight of the cosmetic composition is preferably of from 0.1 to 20 wt. % and in particular preferably of from 1 to 15 wt.-%.

In relation to the total weight thereof, the cosmetic composition includes, as second essential component, 0.01 to 25 wt. % at least of a polyether-modified polysiloxane.

A content of from 0.1 to 15 wt. % of polyether-modified polysiloxanes in the cosmetic compositions is preferred, a content of from 0.2 to 10 wt. % is particularly preferred and a content of from 1 to 5 wt. % is in particular preferred.

Suitable polyether-modified polysiloxanes are preferably understood to mean compounds of the subsequent formula (I)

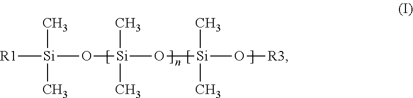

in which
the residues R1 and R3, independently of one another, represent a hydrogen atom, an alkyl group with 1 to 30 C atoms, an alkoxy group with 1 to 30 C atoms or an optionally substituted phenyl group,
the residue R2 represents the group —C_{c}H_{2c}—O—(C₂H₄O—)ₐ(C₃H₆O—)_{b}R5,
the residue R5 represents a hydrogen atom or a linear or branched alkyl group with 1 to 16 C atoms,
n represents a number of from 0-500,
p represents a number of from 1 to 50,
a represents a number of from 0 to 50,
b represents a number of from 0 to 50,
a+b are at least 1, and
c represents a number of from 1 to 4.

More preferred polyether-modified polysiloxane compounds of the general structural formula (I) are:

TABLE 1

| | R1, R3 | R2 | R5 | n | p | a | b | c |
|---|---|---|---|---|---|---|---|---|
| 1 | H, | —C_{c}H_{2c}—O—(C₂H₄O)ₐ—(C₃H₆O)_{b}—R5 | H, | 1-500 | 5-50 | 15-25 | 15-25 | 3 |
| | Methyl | | Methyl | | | | | |
| 2 | H, | —C_{c}H_{2c}—O—(C₂H₄O)ₐ—(C₃H₆O)_{b}—R5 | H, | 1-500 | 5-50 | 5-20 | 0.1 | 3 |
| | Methyl | | Methyl | | | | | |
| 3 | H, | —C_{c}H_{2c}—O—(C₂H₄O)ₐ—(C₃H₆O)_{b}—R5 | H, | 0.1 | 1-3 | 5-10 | 0.1 | 2 |
| | Methyl | | Methyl | | | | | |
| 4 | H, | —C_{c}H_{2c}—O—(C₂H₄O)ₐ—(C₃H₆O)_{b}—R5 | H, | 1-500 | 5-50 | 20-30 | 20-30 | 3 |
| | Methyl | | Methyl | | | | | |
| 5 | H, | —C_{c}H_{2c}—O—(C₂H₄O)ₐ—(C₃H₆O)_{b}—R5 | H, | 1-500 | 5-50 | 10-25 | 10-25 | 3 |
| | Methyl | | Methyl | | | | | |
| 6 | H, | —C_{c}H_{2c}—O—(C₂H₄O)ₐ—(C₃H₆O)_{b}—R5 | H, | 1-500 | 2-50 | 10-30 | 2-10 | 3 |
| | Methyl | | Methyl | | | | | |
| 7 | H, | —C_{c}H_{2c}—O—(C₂H₄O)ₐ—(C₃H₆O)_{b}—R5 | H, | 1-500 | 2-50 | 10-30 | 1-10 | 3 |
| | Methyl | | Methyl | | | | | |

Particularly preferred polyether-modified polysiloxane compounds of the general structural formula (I) are:

TABLE 2

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R5 | H, | 5-500 | 7-50 | 15-20 | 15-20 | 3 |
| | | | Methyl | | | | | |
| 2 | Methyl | —C_{c}H_{2c}—O—(C₂H₄O)ₐ—(C₃H₆O)_{b}—R5 | H, | 5-500 | 7-50 | 7-15 | 0 | 3 |
| | | | Methyl | | | | | |
| 3 | Methyl | —C_{c}H_{2c}—O—(C₂H₄O)ₐ—(C₃H₆O)_{b}—R5 | H, | 0.1 | 1 | 6-8 | 0 | 2 |
| | | | Methyl | | | | | |
| 4 | Methyl | —C_{c}H_{2c}—O—(C₂H₄O)ₐ—(C₃H₆O)_{b}—R5 | H, | 5-500 | 7-50 | 20-25 | 20-25 | 3 |
| | | | Methyl | | | | | |
| 5 | Methyl | —C_{c}H_{2c}—O—(C₂H₄O)ₐ—(C₃H₆O)_{b}—R5 | H, | 5-500 | 7-50 | 15-20 | 15-20 | 3 |
| | | | Methyl | | | | | |
| 6 | Methyl | —C_{c}H_{2c}—O—(C₂H₄O)ₐ—(C₃H₆O)_{b}—R5 | H, | 5-500 | 2-50 | 15-25 | 2-8 | 3 |
| | | | Methyl | | | | | |
| 7 | Methyl | —C_{c}H_{2c}—O—(C₂H₄O)ₐ—(C₃H₆O)_{b}—R5 | H, | 5-500 | 2-50 | 10-15 | 1-5 | 3 |
| | | | Methyl | | | | | |

In particular preferred polyether-modified polysiloxane compounds of the general structural formula (I) are:

TABLE 3

| | R1, R3 | R2 | R5 | n | p | a | b | c |
|---|---|---|---|---|---|---|---|---|
| 1 | Methyl | —C_{c}H_{2c}—O—(C₂H₄O)ₐ—(C₃H₆O)_{b}—R5 | Methyl | 10-500 | 10-50 | 18 | 18 | 3 |
| 2 | Methyl | —C_{c}H_{2c}—O—(C₂H₄O)ₐ—(C₃H₆O)_{b}—R5 | Methyl | 10-500 | 10-50 | 12 | 0 | 3 |
| 3 | Methyl | —C_{c}H_{2c}—O—(C₂H₄O)ₐ—(C₃H₆O)_{b}—R5 | Methyl | 10-500 | 10-50 | 14 | 0 | 3 |
| 4 | Methyl | —C_{c}H_{2c}—O—(C₂H₄O)ₐ—(C₃H₆O)_{b}—R5 | Methyl | 0 | 1 | 7 | 0 | 2 |
| 5 | Methyl | —C_{c}H_{2c}—O—(C₂H₄O)ₐ—(C₃H₆O)_{b}—R5 | Methyl | 10-500 | 10-50 | 22 | 24 | 3 |
| 6 | Methyl | —C_{c}H_{2c}—O—(C₂H₄O)ₐ—(C₃H₆O)_{b}—R5 | Methyl | 10-500 | 10-50 | 17 | 18 | 3 |
| 7 | Methyl | —C_{c}H_{2c}—O—(C₂H₄O)ₐ—(C₃H₆O)_{b}—R5 | Methyl | 10-500 | 5-50 | 20 | 6 | 3 |
| 8 | Methyl | —C_{c}H_{2c}—O—(C₂H₄O)ₐ—(C₃H₆O)_{b}—R5 | Methyl | 10-500 | 5-50 | 14 | 4 | 3 |

Polyether-modified polysiloxane compounds listed in the above-named tables 1-3 are commercially available, for example under the trade names
1: Dow Corning 190 (INCI: PEG/PPG-18/18 Dimethicone),
2: Dow Corning 193 (INCI: PEG-12 Dimethicone),
3: ABIL® B 8843 (INCI: PEG-14 Dimethicone),
4: Silwet L-77,
5: Mirasil DCMO (INCI: Cyclomethicone, PEG/PPG-22/24 Dimethicone),
6: Dow Corning Q2-5220 (INCI: PEG/PPG-17/18 Dimethicone),
7: ABIL® B 88184 (INCI: PEG/PPG-20/6 Dimethicone), and
8: ABIL® B 8851 (INCI: PEG/PPG-14/4 Dimethicone).

In particular, preferred cosmetic compositions are characterized in that they include at least one polyether-modified polysiloxane compound according to formula (I) which meets the conditions of line 2 (tables 1-3) (for example Dow Corning 193 (INCI: PEG-12 Dimethicone)).

Likewise, in particular preferred cosmetic compositions are characterized in that they include at least one polyether-modified polysiloxane compound according to formula (I) which meets the conditions of line 2 in tables 1 and 2 and line 3 in table 3 (for example ABIL® B 8843 (INCI: PEG-14 Dimethicone)).

The use of polyether-modified polysiloxane compounds instead of for example polydimethylsiloxanes leads to transparent cosmetic compositions.

In relation to the total weight thereof, the cosmetic composition includes, as third essential component, a film-forming polymer. The cosmetic composition can also include several film-forming polymers. Film-forming polymers are understood to mean such polymers which leave a continuous film on the hair upon drying. Film-forming polymers are also understood to mean such polymers which, upon application in 0.01 to 20 wt. % aqueous, alcohol or aqueous-alcohol solution, are capable of depositing a transparent polymer film on the hair.

Permanent and also temporary cationic, anionic, non-ionic or amphoteric polymers are also permanently suitable as film-forming polymers. The film-forming polymers can be of synthetic or natural origin.

Suitable preferably used synthetic, film-forming polymers are homo- or copolymers which are made from at least one of the following monomers: vinylpyrrolidone, vinylcaprolactam, vinylester such as for example vinylacetate, vinylalcohol, acrylamide, methacrylamide, C1 to C7 alkyl acrylamide, C1 to C7 dialkyl acrylamide, C1 to C7 alkyl methacrylamide, C1 to C7 dialkyl methacrylamide, C 1 to C7 alkyl acrylate, acrylic acid, propylene glycol, ethylene glycol, wherein the C1 to C7 alkyl groups of these monomers are preferably C1 to C3 alkyl groups. Homopolymers of the vinylcaprolactam, vinylpyrrolidone or N-vinyl formamide are named by way of example. Further synthetic film-forming, hair-setting polymers are for example copolymerizates of vinylpyrrolidone and vinylacetate, vinylpyrrolidone and styrene or terpolymers of vinylpyrrolidone, vinylacetate and vinyl propionate, polyacrylamide. Suitable natural film-forming polymers are for example cellulose derivatives, for example hydroxypropyl cellulose with a molecular weight of from 30,000 to 50,000 g/mol.

Further examples of film-forming polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/lsophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C 1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/ltaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/lsophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer.

It is preferred that the film-forming polymer is a non-ionic, film-forming polymer. It is in particular preferred that the cosmetic compositions include polyvinylpyrrolidone (PVP) and/or a vinylpyrrolidone-containing copolymer as film-forming polymer. Suitable polyvinylpyrrolidones can for example be obtained under the name Luviskol® K from BASF SE. Of the vinylpyrrolidone-containing copolymers a styrene/VP copolymer and/or a vinylpyrrolidone-vinylacetate copolymer and/or a VP/DMAPA Acrylates copolymer and/or a VP/Vinyl Caprolactam/DMAPA Acrylates Copolymer are quite particularly preferably used in the cosmetic compositions.

Vinylpyrrolidone-vinylacetate copolymers are marketed under the name Luviskol® VA from BASF SE. A VP/Vinyl Caprolactam/DMAPA Acrylates Copolymer is marketed for example under the trade name Aquaflex® SF-40 from Ashland Inc. A VP/DMAPA Acrylates Copolymer are marketed for example under the name Styleze CC-10 from Ashland and is a highly preferred vinylpyrrolidone-containing copolymer.

It may be preferred, in particular when using poly(methacryloyloxyethyl trimethylammonium chloride) as conditioning polymer, that the cosmetic composition includes polyvinylpyrrolidone and VP/DMAPA Acrylates Copolymer as film-forming polymer.

The cosmetic compositions include the film-forming polymer preferably in quantities of up to 15.0 wt.-%, preferably in quantities of up to 10.0 wt. % and quite particularly preferably in quantities of between 1 and 7.5 wt.-%, each in relation to the total weight thereof.

Hereinafter, further constituents of the cosmetic compositions are described which can be included in the compositions in addition to the above-described necessary ingredients.

Further suitable ingredients include surfactants, lipid substances, waxes, protein hydrolyzates, amino acids, oligopeptides, vitamins, provitamins, vitamin precursors, betaines, bioquinones, purine (derivatives), taurine (derivatives), plant extracts, silicones, ester oils, UV light protection filters, structuring agents, thickeners, electrolytes, pH suspending agents, swelling agents, dyes, antidandruff active ingredients, complexers, opacifiers, pearlescing agents, pigments, stabilizing agents, propellants, antioxidants, perfume oils and/or preservatives.

The cosmetic agents preferably also include at least one representative from the group of anionic, amphoteric, zwitterionic, non-ionic, cationic surfactants or mixtures thereof. Emulsifiers which are solid at room temperature (20° C.), in particular non-ionic emulsifiers which are solid at room temperature (20° C.), are preferably used.

In particular the fatty acid partial glycerides, including monoglycerides, diglycerides and the technical mixtures thereof, are understood to be suitable as non-ionic emulsifiers. Typical examples are mono- and/or diglycerides based on hexanoic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid as well as the technical mixtures thereof. Preferably oleic acid monoglycerides are used. The surface-active, medium-chain fatty alcohols, in particular cetyl alcohol and stearyl alcohol, form a further group of preferred emulsifiers.

At least one cationic surfactant selected from quaternary ammonium compounds, esterquats, amidoamines or mixtures thereof can preferably be used. Preferred quaternary ammonium compounds are ammonium halogenides, in particular chlorides and bromides, such as alkyl trimethyl ammonium chlorides, dialkyl dimethyl ammonium chlorides and trialkyl methyl ammonium chlorides. The long alkyl chains of these surfactants preferably have 10 to 18 carbon atoms, such as for example in cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Further preferred cationic surfactants are the imidazolium compounds known under INCI names Quaternium-27 and Quaternium-83.

The additional surfactants are included in the compositions preferably in a quantity of from 0.01 wt. % to 5 wt.-%, particularly preferably of from 0.1 wt. % to 2.5 wt.-%, in each case in relation to the weight of the composition.

The cosmetic compositions can additionally include an organic polyol. Preferred compositions include the organic polyol in relation to the total weight thereof in quantities of from 0.5 to 60, preferably 1.0 to 50 wt. % and in particular 1.5 to 25 wt.-%.

The polyol can be used as an individual substance or in the form of polyol mixtures. Preferred cosmetic agents are characterized in that they include fewer than four, preferably one to three, but in particular only one or two polyol(s). In particular polyols from the group glycerol, 1,2-ethanediol, polyethylene glycols with MW>400, propane diol, butane diol, in particular 1,3-butanediol, hexanediol, in particular 1,6-hexanediol, sorbitol, threitol, erythritol, arabitol, altritol, ribitol, xylitol, galactitol, mannitol, iditol and panthenol are suitable for producing cosmetic compositions. Particularly suitable organic polyols are glycerol, sorbitol and panthenol. Preferred cosmetic compositions are characterized in that they include at least one polyol from the group glycerol, polyethylene glycol, propylene glycol, butylene glycol and hexanediol, preferably glycerol and/or propylene glycol, as organic polyol.

The addition of a polyol can improve the properties of the cosmetic compositions in storage and application, and has advantageous cosmetic effects.

The percentage by weight of a polyol in the total weight of the cosmetic compositions can vary in wide ranges. In cosmetic compositions with a high polyol content, preferably a smaller quantity of water is added to the compositions in compensation.

The water content of the cosmetic compositions is preferably at least 10 wt.-%, preferably at least 40 wt. % and in particular preferably from 50 to 90 wt.-%, in each case in relation to the total weight of the cosmetic composition.

Preferred hair-treatment agents are characterized as follows:
a) 0.1 to 20 wt. % of a mixture of at least two conditioning polymers, wherein a conditioning polymer is selected from the group consisting of cationic guar compounds and poly(methacryloyloxyethyl trimethylammonium chloride),
b) 0.2 to 5 wt. % of a polyether-modified polysiloxane,
c) 1.0 to 7.5 wt. % of a film-forming polymer and
d) 50 to 90 wt. % water.

More preferred hair-treatment agents are furthermore characterized as follows:
a) 1 to 15 wt. % of a mixture of at least two conditioning polymers, wherein a conditioning polymer is selected from the group consisting of cationic guar compounds and poly(methacryloyloxyethyl trimethylammonium chloride),
b) 0.2 to 5 wt. % of a polyether-modified polysiloxane which is known under INCI name PEG-12 Dimethicone or PEG-14 Dimethicone,
c) 1.0 to 7.5 wt. % of a film-forming polymer and
d) 50 to 90 wt. % water.

Even more preferred hair-treatment agents are characterized as follows:
a) 1 to 15 wt. % of a mixture of at least two conditioning polymers, comprising poly(methacryloyloxyethyl trimethylammonium chloride) and a compound selected from the group consisting of polymers which are known under INCI names Polyquaternium-11, Polyquaternium-46 and Polyquaternium-72, as well as mixtures thereof,
b) 0.2 to 5 wt. % of a polyether-modified polysiloxane which is known under INCI name PEG-12 Dimethicone or PEG-14 Dimethicone,
c) 1.0 to 7.5 wt. % of a film-forming polymer and
d) 50 to 90 wt. % water.

Alternative, even more preferred hair-treatment agents are characterized as follows:
a) 1 to 15 wt. % of a mixture of at least two conditioning polymers, comprising a cationic guar compound and a compound selected from the group consisting of polymers which are known under INCI names Polyquaternium-11, Polyquaternium-46 and Polyquaternium-72, as well as mixtures thereof,
b) 0.2 to 5 wt. % of a polyether-modified polysiloxane which is known under INCI name PEG-12 Dimethicone or PEG-14 Dimethicone,
c) 1.0 to 7.5 wt. % of a film-forming polymer and
d) 50 to 90 wt. % water.

Quite particularly preferred hair-treatment agents are characterized as follows:
a) 1 to 15 wt. % of a mixture of at least two conditioning polymers, comprising poly(methacryloyloxyethyl trimethylammonium chloride) and a compound which is known under INCI name Polyquaternium-72,
b) 0.2 to 5 wt. % of a polyether-modified polysiloxane which is known under INCI name PEG-12 Dimethicone or PEG-14 Dimethicone,
c) 1.0 to 7.5 wt. % of a film-forming polymer selected from the group consisting of polyvinylpyrrolidone (PVP), a vinylpyrrolidone-containing copolymer and mixtures thereof, and
d) 50 to 90 wt. % water.

Likewise quite particularly preferred hair-treatment agents are characterized as follows:
a) 1 to 15 wt. % of a mixture of at least two conditioning polymers, comprising a cationic guar compound and a compound which is known under INCI name Polyquaternium-11,
b) 0.2 to 5 wt. % of a polyether-modified polysiloxane which is known under INCI name PEG-12 Dimethicone or PEG-14 Dimethicone,
c) 1.0 to 7.5 wt. % of a film-forming polymer selected from the group consisting of polyvinylpyrrolidone (PVP), a vinylpyrrolidone-containing copolymer and mixtures thereof, and
d) 50 to 90 wt. % water.

Highly preferred hair-treatment agents are characterized as follows:
a) 1 to 15 wt. % of a mixture of at least two conditioning polymers, comprising poly(methacryloyloxyethyl trimethylammonium chloride) and a compound which is known under INCI name Polyquaternium-72,
b) 0.2 to 5 wt. % of a polyether-modified polysiloxane which is known under INCI name PEG-12 Dimethicone,
c) 1.0 to 7.5 wt. % of a mixture of two film-forming polymers, comprising polyvinylpyrrolidone (PVP) and a polymer which is known under INCI name VP/DMAPA Acrylates Copolymer, and
d) 50 to 90 wt. % water.

Likewise highly preferred hair-treatment agents are characterized as follows:
a) 1 to 15 wt. % of a mixture of at least two conditioning polymers, comprising a cationic guar compound and a compound which is known under INCI name Polyquaternium-11,
b) 0.2 to 5 wt. % of a mixture of polyether-modified polysiloxanes which are known under INCI names PEG-12 Dimethicone and PEG-14 Dimethicone,
c) 1.0 to 7.5 wt. % of a polymer which is known under INCI name VP/DMAPA Acrylates Copolymer, and
d) 50 to 90 wt. % water.

A second subject matter of this application is the use of a cosmetic composition according to the invention for temporary shaping of keratin-containing fibers, in particular human hair. The human hair is highly preferably curly human hair.

A third subject matter of this application is the use of a cosmetic composition according to the invention for improving at least one of the following properties of curly hair:
curl retention,
curl bounce,
curl separation,
shine and
styleability.

The cosmetic compositions according to the invention can be used in a method for temporary shaping of keratin-containing fibers, in particular human hair, in which the keratin fibers are loaded with a cosmetic composition and temporarily fixed in terms of shape. The human hair is highly preferably curly human hair. Preferably, the cosmetic composition is applied to shampooed and hand-dried hair and the hair is then air-dried or dried with heat, for example an electric hairdryer, curling tongs or heating hoods, optionally using shaping agents such as hair rollers or papillotes.

In respect of the preferred embodiments of the use or method, the same applies, mutatis mutandis, to that which has been said about the compositions.

The composition of some preferred cosmetic compositions A to E can be seen in the following tables (details in wt. % in relation to the total weight of the cosmetic agent unless otherwise indicated).

| Ingredient | A | B | C | D | E |
|---|---|---|---|---|---|
| Cetrimonium Chloride | 0.2 | 0.2 | 0.2 | 2 | 2 |
| Guar Hydroxypropyltrimonium | 1.2 | 1.7 | 1.6 | — | — |
| Chloride | | | | | |
| Polyquaternium-37 | — | — | — | 2 | 2 |
| Polyquaternium-11 | 6 | 6 | 7 | — | — |
| Polyquaternium-46 | — | 3 | 4 | — | — |
| Polyquaternium-72 | — | — | — | 5 | 5 |
| PEG-12 Dimethicone | 0.2 | 1 | 1 | 1 | 1 |
| PEG-14 Dimethicone | — | 1 | 1 | — | — |
| PVP | — | — | — | 1.5 | 1.5 |
| VP/DMAPA Acrylates | 4.3 | 3 | 4 | 1.5 | 5 |
| Copolymer | | | | | |
| Glycerol | — | — | — | 7.5 | 7.5 |
| Propylene Glycol | — | — | — | 7.5 | 7.5 |
| Perfume | 0.2 | 0.2 | 0.2 | 0.25 | 0.25 |
| Hydrogenated Castor Oil | 0.5 | 0.5 | 0.5 | 0.5 | 0.5 |
| Citric Acid | 0.2 | 0.2 | 0.2 | — | — |
| Panthenol | 0.25 | 0.25 | 0.25 | 0.2 | 0.2 |
| Water, Preservative and | up to 100 | up to | up to | up to | up to |
| Accompanying Substances | | 100 | 100 | 100 | 100 |

Formulations A to E are transparent compositions with pleasant sensory properties. Thus when applied by hand they do not leave behind a greasy/slimy sensation either on the hand itself or on the wet hair treated therewith.

Formulations A to E give hair treated therewith easy styleability, high shine, healthy appearance and, if curly, a long-lasting curly appearance without making the hair sticky or stiff.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A transparent cosmetic composition, comprising, in relation to the total weight thereof,
a) 0.1 to 30 wt. % of a mixture of at least two conditioning polymers, wherein at least one of the conditioning polymer is selected from the group consisting of cationic guar compounds and poly(methacryloyloxyethyl trimethylammonium chloride) and mixtures thereof,
b) 0.01 to 25 wt. % of a polyether-modified polysiloxane,
c) 0.001 to 15 wt. % of a film-forming polymer, and
d) 1 to 90 wt. % water.

2. The transparent cosmetic composition according to claim 1, wherein in that the film-forming polymer is a non-ionic film-forming polymer.

3. The transparent cosmetic composition according to claim 1, wherein the film-forming polymer is selected from the group consisting of: polyvinylpyrrolidone (PVP), a vinylpyrrolidone-containing copolymer, and mixtures thereof.

4. The transparent cosmetic composition according to claim 1, wherein the film-forming polymer comprises a mixture of polyvinylpyrrolidone (PVP) and a polymer which is known under INCI name VP/DMAPA Acrylates Copolymer.

5. The transparent cosmetic composition according to claim 1, wherein one of the at least two conditioning polymers is selected from the group consisting of: polyquaternium-11, polyquaternium-46, polyquaternium-72, as well as mixtures thereof.

6. The transparent cosmetic composition according to claim 1, wherein the mixture of conditioning polymers comprises poly(methacryloyloxyethyl trimethylammonium chloride) and polyquaternium-72.

7. The transparent cosmetic composition according to claim 1, further comprising a cationic surfactant.

8. The transparent cosmetic composition according to claim 1, further comprising an organic polyol.

9. A method of improving a styling property for human hair, comprising:
applying the composition of claim 1 to human hair, wherein the styling property is selected from the group consisting of: curl retention, curl bounce, curl separation, shine, styleability, and combinations thereof.
